(19) United States
(12) Patent Application Publication
(10) Pub. No.: US 2008/0145405 A1
(43) Pub. Date: Jun. 19, 2008

(54) DRUG DELIVERY DEVICES

(76) Inventors: Jay F. Kunzler, Canandaigua, NY (US); Dharmendra M. Jani, Fairport, NY (US)

Correspondence Address: Bausch & Lomb Incorporated, One Bausch & Lomb Place, Rochester, NY, 14604-2701, US

(21) Appl. No.: 11/611,674

(22) Filed: Dec. 15, 2006

(51) Int. Cl. A61F 9/00

(52) U.S. Cl. 424/427

(57) ABSTRACT

A method for making an ocular drug delivery device, the method comprising providing a drug delivery device comprising a core comprising a therapeutically effective amount of one or more pharmaceutically active agents and a first polymeric material, and a shell covering the core, the shell comprising a second polymeric material which is permeable to passage of the active agent, wherein the first and/or second polymeric material include one or more contaminants and wherein the drug delivery device is sized and configured for implantation or injection in eye tissue; and subjecting the drug delivery device to a supercritical fluid to remove the contaminants.

### BACKGROUND OF THE INVENTION

1. Technical Field

The present invention generally relates to drug delivery devices for ocular drug delivery, such as a device placed or implanted in the eye to release a pharmaceutically active agent to the eye, and methods for making such devices.

2. Description of Related Art

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placement in the eye and treatment of various eye diseases. See, e.g., U.S. Pat. Nos. 5,378,475; 5,773,019; 5,902,598; 6,001,386; 6,217,895; 6,375,972; 6,756,049; and 6,756,058; and U.S. Patent Application Publication Nos. 2002/0086051 A1; 2002/0106395 A1; 2002/0110635 A1; 2004/0265356 A1; and 2005/0261668. Many of these devices contain a pharmaceutically active agent and a polymeric material, such as silicone or other hydrophobic materials. As an example, such devices may include an inner drug core including the active agent mixed with a permeable polymeric material, and some type of holder made of a polymeric material impermeable to passage of the active agent. Another example is a matrix of the active agent and a polymeric material.

Various prior methods of making these types of devices involve the step of extracting the polymeric material to remove impurities such as unreacted monomers or oligomers therefrom. The extraction process is important to ensure the device does not leach such impurities once introduced to eye tissue. Extraction is especially important for silicone polymeric materials, as unreacted monomers or oligomers of silicone may be non-biocompatible (for example, irritating to eye tissue or even toxic). A typical method of extracting such polymers employs isopropanol, or other liquid polar solvents, as the extracting material. Accordingly, it is necessary to perform the extraction prior to combining the polymeric material with the active agent.

U.S. Patent Application Publication No. 2006/0078592 A1 (“the '592 application”) discloses a method for making an ocular drug delivery device which involves providing a drug delivery device comprising a polymeric material and a pharmaceutically active agent, the polymeric material including contaminants, and the drug delivery device being sized and configured for implantation or injection in eye tissue; and subjecting the device to a supercritical fluid to remove the contaminants. The '592 application further discloses that the device includes a holder for the inner drug core wherein the holder is made of a material that is impermeable to passage of the active agent therethrough, e.g., a silicone material such as a polydimethylsiloxane material, and having at least one passageway therein to permit the active agent to pass therethrough and contact eye tissue.

It would be desirable to provide improved drug delivery devices which contain a relatively low content of contaminants after extraction of the device.

### SUMMARY OF THE INVENTION

In accordance with a first embodiment of the present invention, a method for making an ocular drug delivery device is provided comprising providing a drug delivery device comprising (a) a core comprising a therapeutically effective amount of one or more pharmaceutically active agents and a first polymeric material, and (b) a shell covering the core, the shell comprising a second polymeric material which is permeable to passage of the one or more pharmaceutically active agents, wherein the first and/or second polymeric material include one or more contaminants and wherein the drug delivery device is sized and configured for implantation or injection in eye tissue; and subjecting the drug delivery device to a supercritical fluid to remove the contaminants.

In accordance with a second embodiment of the present invention, a method for making an ocular drug delivery device is provided comprising providing a drug delivery device comprising (a) a core comprising a therapeutically effective amount of one or more pharmaceutically active agents and a first polymeric material, and (b) a shell covering the core, the shell comprising a second polymeric material which is permeable to passage of the one or more pharmaceutically active agents; and removing contaminants from the device by subjecting the device to a supercritical fluid.

### BRIEF DESCRIPTION OF THE DRAWINGS

FIGS. 1A-1K are a side view depicting one embodiment for preparing a drug delivery device of the present invention.

FIGS. 2A-2O are a side view depicting a second embodiment for preparing a drug delivery device of the present invention.

### DETAILED DESCRIPTION OF THE PREFERRED EMBODIMENTS

The drug delivery devices of the present invention include at least a core containing at least a therapeutically effective amount of one or more pharmaceutically active agents and a first polymeric material and a shell covering the core, the shell formed from a second polymeric material which is permeable to passage of the one or more pharmaceutically active agents.

Generally, pharmaceutically active agents or drugs useful in the drug delivery devices of the present invention can be any compound, composition of matter, or mixtures thereof that can be delivered from the device to produce a beneficial and useful result to the eye, especially an agent effective in obtaining a desired local or systemic physiological or pharmacological effect. Examples of such agents include, but are not limited to, anesthetics and pain killing agents such as lidocaine and related compounds, benzodiazepam and related compounds and the like; anti-cancer agents such as 5-fluorouracil, adriamycin and related compounds and the like; anti-fungal agents such as fluconazole and related compounds and the like; anti-viral agents such as trisodium phosphomonoformate, trifluorothymidine, acyclovir, ganciclovir, DDI, AZT and the like; cell transport/mobility impending agents such as colchicine, vincristine, cytochalasin B and related compounds and the like; antiglaucoma drugs such as beta-blockers, e.g., timolol, betaxolol, atenalol, and the like; antihypertensives; decongestants such as phenylephrine, naphazoline, tetrahydrazoline and the like; immunological response modifiers such as muramyl dipeptide and related compounds and the like; peptides and proteins such as cyclosporin, insulin, growth hormones, insulin related growth factor, heat shock proteins and related compounds and the like; steroidal compounds such as dexamethasone, prednisolone and related compounds and the like; low solubility steroids such as fluocinolone acetonide and related compounds and the like; carbonic anhydrase inhibitors; diagnostic agents; antiapoptosis agents; gene therapy agents; sequestering agents; reductants such as glutathione and the like; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen and the like; nutrients and vitamins: enzyme inhibitors: antioxidants; anticataract drugs; aldose reductase inhibitors; cytoprotectants; cytokines, cytokine inhibitors, and cytokin protectants; uv blockers; mast cell stabilizers; anti neovascular agents such as antiangiogenic agents, e.g., matrix metalloprotease inhibitors and the like.

Representative examples of additional pharmaceutically active agents for use herein include, but are not limited to, neuroprotectants such as nimodipine and related compounds and the like; antibiotics such as tetracycline, chlortetracycline, bacitracin, neomycin, polymyxin, gramicidin, oxytetracycline, chloramphenicol, gentamycin, erythromycin and the like; anti-infectives; antibacterials such as sulfonamides, sulfacetamide, sulfamethizole, sulfisoxazole; nitrofurazone, sodium propionate and the like; antiallergenics such as antazoline, methapyriline, chlorpheniramine, pyrilamine, prophenpyridamine and the like; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triminolone and the like; miotics; anti-cholinesterase such as pilocarpine, eserine salicylate, carbachol, di-isopropyl fluorophosphate, phospholine iodine, demecarium bromide and the like; miotic agents; mydriatics such as atropine sulfate, cyclopentolate, homatropine, scopolamine, tropicamide, eucatropine, hydroxyamphetamine and the like; svmpathomimetics such as epinephrine and the like; and prodrugs such as, for example, those described in Design of Prodrugs, edited by Hans Bundgaard, Elsevier Scientific Publishing Co., Amsterdam, 1985. In addition to the foregoing agents, other agents suitable for treating, managing, or diagnosing conditions in a mammalian organism may be entrapped in the copolymer and administered using the drug delivery systems of the current invention. Once again, reference may be made to any standard pharmaceutical textbook such as, for example, Remington's Pharmaceutical Sciences for pharmaceutically active agents.

Any pharmaceutically acceptable form of the foregoing pharmaceutically active agents may be employed in the practice of the present invention, e.g., the free base; free acid; pharmaceutically acceptable salts, esters or amides thereof, e.g., acid additions salts such as the hydrochloride, hydrobromide, sulfate, bisulfate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulfate salts and the like; alkali or alkaline earth metal salts such as the sodium, calcium, potassium and magnesium salts and the like; hydrates; solvates, enantiomers; isomers; stereoisomers; diastereoisomers; tautomers; polymorphs, mixtures thereof, prodrugs thereof or racemates or racemic mixtures thereof.

Actual dosage levels of the pharmaceutically active agent(s) in the drug delivery devices of the present invention may be varied to obtain an amount of the pharmaceutically active agent(s) that is effective to obtain a desired therapeutic response for a particular system and method of administration. The selected dosage level therefore depends upon such factors as, for example, the desired therapeutic effect, the route of administration, the desired duration of treatment, and other factors. The total daily dose of the pharmaceutically active agent(s) administered to a host in single or divided doses can vary widely depending upon a variety of factors including, for example, the body weight, general health, sex, diet, time and route of administration, rates of absorption and excretion, combination with other drugs, the severity of the particular condition being treated, etc. Generally, the amounts of pharmaceutically active agent(s) present in the drug delivery systems of the present invention can range from about 1% w/w to about 60% w/w and preferably from about 5% w/w to about 50% w/w.

In addition to the illustrated materials below, a wide variety of materials may be used as a first polymeric material for forming the core containing the pharmaceutically active agents of the drug delivery devices of the present invention. The only requirements are that they are inert, non-immunogenic, of the desired permeability, and capable of being cut into shaped articles. Materials that may be suitable for fabricating the core of the device include naturally occurring or synthetic materials that are biologically compatible with body fluids and body tissues, and essentially insoluble in the body fluids with which the material will come in contact and capable of being cut into shaped articles.

In one embodiment, exemplary polymeric materials include those prepared by polymerizing a monomeric mixture containing at least one or more hydrophilic monomers and optionally a hydrophobic monomer and crosslinking agent. Examples of hydrophobic monomers useful for copolymerization include, but are not limited to, N,N-dimethylacrylamide, N-methylacrylamide and the like, with N,N-dimethylacrylamide being preferred for increased hydrophilicity. Additional hydrophilic monomers for use herein include, but are not limited to, unsaturated carboxylic acids, e.g., acrylic acids, methacrylic acids and the like; (meth)acrylic substituted alcohols, e.g., 2-hydroxyethyl methacrylate, 2-hydroxyethyl acrylate and the like; vinyl lactams, e.g., N-vinyl pyrrolidones and the like. Further additional hydrophilic monomers for use herein include the vinyl carbonate monomers, vinyl carbamate monomers and the oxazolone monomersdisclosed in U.S. Pat. No. 4,910,277. Other suitable hydrophilic monomers will be apparent to one skilled in the art. Mixtures of the foregoing hydrophilic monomers are also contemplated.

Useful hydrophobic monomers for use herein include, but are not limited to, cycloalkyl acrylates and methacrylates, e.g., tert-butyl cyclohexyl methacrylate, isopropylcyclopentyl acrylate, tert-butylcyclohexyl acrylate and the like; siloxysilane monomers, 2-ethylhexyl methacrylate, 2-phenyloxyethyl methacrylate and the like and mixtures thereof.

Useful crosslinking agents include, but are not limited to, diacrylates and dimethacrylates of triethylene glycol, butylene glycol, neopentyl glycol, ethylene glycol, hexane-1,6-diol and thio-diethylene glycol; trimethylolpropane triacrylate, N,N′-dihydroxyethylene bisacrylamide, diallyl phthalate, triallyl cyanurate, divinylbenzene, ethylene glycol divinyl ether, N,N′-methylene-bis-(meth)acrylamide, sulfonated divinylbenzene, divinylsulfone and the like and mixtures thereof.

In another embodiment, exemplary polymeric materials include those prepared by polymerizing a monomeric mixture containing at least one or more acrylate ester and/or methacrylate ester-containing monomers or prepolymers and one or more acrylamido-containing monomers optionally in the presence of one or more crosslinking agents. The resulting copolymers can be in random or block sequences.

Suitable acrylate ester and/or methacrylate ester-containing monomers may be represented by the general formula:

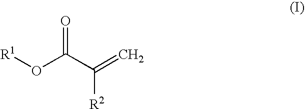

wherein R¹ may be a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, C₁-C₁₈ alkyl siloxysilane, C₁-C₁₈ alkyl siloxane, ether or polyether containing groups, substituted or unsubstituted, linear or branched, and R² is H or CH₃.

Representative examples of alkyl groups for use herein include, by way of example, a straight or branched hydrocarbon chain radical containing carbon and hydrogen atoms of from 1 to about 18 carbon atoms with or without unsaturation, to the rest of the molecule, e.g., methyl, ethyl, n-propyl, 1-methylethyl (isopropyl), n-butyl, n-pentyl and the like.

Representative examples of cycloalkyl groups for use herein include, by way of example, a substituted or unsubstituted non-aromatic mono or multicyclic ring system of about 3 to about 18 carbon atoms such as, for example, cyclopropyl, cyclobutyl, cyclopentyl, cyclohexyl, perhydronapththyl, adamantyl and norbomyl groups bridged cyclic group or sprirobicyclic groups, e.g., sprio-(4,4)-non-2-yl and the like, optionally containing one or more heteroatoms, e.g., O and N, and the like.

Representative examples of cycloalkylalkyl groups for use herein include, by way of example, a substituted or unsubstituted cyclic ring-containing radical containing from about 3 to about 18 carbon atoms directly attached to the alkyl group as defined above which is then attached to the main structure of the monomer (via the oxygen atom) at any carbon atom from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like.

Representative examples of cycloalkenyl groups for use herein include, by way of example, a substituted or unsubstituted cyclic ring-containing radical containing from about 3 to about 18 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like.

Representative examples of aryl groups for use herein include, by way of example, a substituted or unsubstituted monoaromatic or polyaromatic radical containing from about 5 to about 25 carbon atoms such as, for example, phenyl, naphthyl, tetrahydronapthyl, indanyl, biphenyl and the like, optionally containing one or more heteroatoms, e.g., O and N, and the like.

Representative examples of arylalkyl groups for use herein include, by way of example, a substituted or unsubstituted aryl group as defined above directly attached to an alkyl group as defined above which is then attached to the main structure of the monomer (via the oxygen atom) at any carbon atom from the alkyl group that results in the creation of a stable structure, e.g., —CH₂C₆H₅, —C₂H₅C₆H₅ and the like, wherein the aryl group can optionally contain one or more heteroatoms, e.g., O and N, and the like.

Representative examples of alkyl siloxysilane groups for use herein include, by way of example, a siloxysilane group directly attached to an alkyl group as defined above which is then attached to the main structure of the monomer (via the oxygen atom) at any carbon atom from the alkyl group that results in the creation of a stable structure, e.g., -(CH₂)h siloxysilane such as one represented by the following structure:

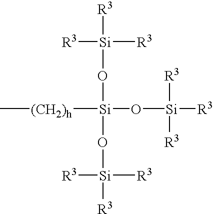

wherein h is 1 to 18 and each R³ independently denotes an lower alkyl radical, phenyl radical or a group represented by

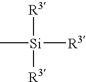

wherein each R^{3′} independently denotes a lower alkyl or aryl radical as defined above. Representative examples of such acrylate ester and/or methacrylate ester-containing monomers include 3-methacryloyloxypropyltris(trimethylsiloxy)silane or tris(trimethylsiloxy)silylpropyl methacrylate, sometimes referred to as TRIS and tris(trimethylsiloxy)silylpropyl vinyl carbamate, sometimes referred to as TRIS-VC and the like and are commercially available from such sources as Gelest, Inc. (Morrisville, PA) and can be prepared by methods well known in the art.

Representative examples of alkyl siloxane groups for use herein include, by way of example, a siloxane group directly attached to an alkyl group as defined above which is then attached to the main structure of the monomer (via the oxygen atom) at any carbon atom from the alkyl group that results in the creation of a stable structure, e.g., —(CH₂)ₓ siloxane such as one represented by the following structure:

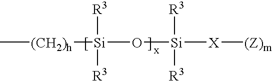

wherein x is an integer from 0 to about 300; h is an integer from 1 to 18, m is an integer from 1 to about 6, each R³ is independently hydrogen, or a lower alkyl or aryl radical as defined above; X is a bond, straight or branched C₁-C₃₀ alkyl group, a C₁-C₃₀ fluoroalkyl group, a substituted or unsubstituted C₅-C₃₀ arylalkyl group, a substituted or unsubstituted C₁-C₃₀ alkoxy group, an ether or polyether containing group, sulfide, or amino-containing group and Z is a polymerizable ethylenically unsaturated organic radical, e.g., (meth)acrylate-containing radicals, (meth)acrylamide-containing radicals, vinylcarbonate-containing radicals, vinylcarbamate-containing radicals, styrene-containing radicals and the like. A representative example of such an acrylate ester and/or methacrylate ester-containing monomer includes α,ω-methacrylate end capped polydimethyl(siloxanes) and the like and are commercially available from such sources as Gelest, Inc. (Morrisville, Pa.) and can be prepared by methods well known in the art.

Representative examples of ether or polyether containing groups for use herein include, by way of example, an alkyl ether, cycloalkyl ether, cycloalkylalkyl ether, cycloalkenyl ether, aryl ether, arylalkyl ether wherein the alkyl, cycloalkyl, cycloalkylalkyl, cycloalkenyl, aryl, and arylalkyl groups are defined above, e.g., alkylene oxides, poly(alkylene oxide)s such as ethylene oxide, propylene oxide, butylene oxide, poly(ethylene oxide)s, poly(ethylene glycol)s, poly(propylene oxide)s, poly(butylene oxide)s and mixtures thereof, an ether or polyether group of the general formula —R⁴OR⁴, wherein R⁴ is a bond, an alkyl, cycloalkyl or aryl group as defined above and R⁴′ is an alkyl, cycloalkyl or aryl group as defined above, e.g., —CH₂CH₂OC₆H₅ and —CH₂CH₂OC₂H₅, and the like.

The substituents in the ‘substituted alkyl’, ‘substituted cycloalkyl’, ‘substituted cycloalkylalkyl’, ‘substituted cycloalkenyl’, ‘substituted arylalkyl’ and ‘substituted aryl’ may be the same or different with one or more selected from the group such as hydrogen, halogen (e.g., fluorine), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted aryl, substituted or unsubstituted heteroaryl, substituted heterocyclylalkyl ring, substituted or unsubstituted heteroarylalkyl, substituted or unsubstituted heterocyclic ring.

In one embodiment, useful acrylate ester or methacrylate ester-containing monomers include, but are not limited to, a linear or branched, substituted or unsubstituted, C₁ to C₁₈ alkyl acrylate, a linear or branched, substituted or unsubstituted, C₁ to C₁₈ alkyl methacrylate, a substituted or unsubstituted C₃ to C₁₈ cycloalkyl acrylate, a substituted or unsubstituted C₃ to C₁₈ cycloalkyl methacrylate, a substituted or unsubstituted C₆ to C₂₅ aryl or alkaryl acrylate, a substituted or unsubstituted C₆ to C₂₅ aryl or alkaryl methacrylate, an ethoxylated acrylate, an ethoxylated methacrylate, partially fluorinated acrylates, partially fluorinated methacrylates and the like and mixtures thereof. In another embodiment, the acrylate ester and/or methacrylate ester-containing monomers are hydrophobic monomers.

Representative examples of acrylate ester-containing monomers for use herein include, but are not limited to, methyl acrylate, ethyl acrylate, propyl acrylate, isopropyl acrylate, n-butyl acrylate, iso-butyl acrylate, t-butyl acrylate, n-hexyl acrylate, 2-ethylbutyl acrylate, 2-ethylhexyl acrylate, cyclopropyl acrylate, cyclobutyl acrylate, cyclohexyl acrylate, benzyl acrylate, 2-phenoxyethyl acrylate, phenyl acrylate, 2-phenylethyl acrylate, 3-phenylpropyl acrylate, 3-phenoxypropyl acrylate, 4-phenylbutyl acrylate, 4-phenoxybutyl acrylate, 4-methylphenyl acrylate, 4-methylbenzyl acrylate, 2-2-methylphenylethyl acrylate, 2-3-methylphenylethyl acrylate, 2-methylphenylethyl acrylate and the like and mixtures thereof.

Representative examples of methacrylate ester-containing monomers for use herein include, but are not limited to, methyl methacrylate, ethyl methacrylate, propyl methacrylate, isopropyl methacrylate, n-butyl methacrylate, iso-butyl methacrylate, t-butyl methacrylate, n-hexyl methacrylate, 2-ethylbutyl methacrylate, 2-ethylhexyl methacrylate, cyclopropyl methacrylate, cyclobutyl methacrylate, cyclohexyl methacrylate, benzyl methacrylate, 2-phenoxyethyl methacrylate, phenyl methacrylate, 2-phenylethyl methacrylate, 3-phenylpropyl methacrylate, 3-phenoxypropyl methacrylate, 4-phenylbutyl methacrylate, 4-phenoxybutyl methacrylate, 4-methylphenyl methacrylate, 4-methylbenzyl methacrylate, 2-2-methylphenylethyl methacrylate, 2-3-methylphenylethyl methacrylate, 2-4-methylphenylethyl methacrylate and the like and mixtures thereof.

Suitable acrylamido-containing monomers may be represented by the general formulae II and III

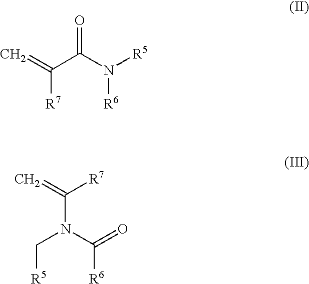

wherein R⁵ and R⁶ are independently hydrogen, a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, C₁-C₁₈ alkyl siloxysilane or C₁-C₁₈ alkyl siloxane, substituted or unsubstituted, linear or branched, as defined above or R₅ and R⁶ together with the nitrogen atom to which they are bonded are joined together to form a heterocyclic group and R⁷ is H or CH₃.

Representative examples of acrylamido-containing monomers include, but are not limited to, acrylamide, N-methylacrylamide, N-ethylacrylamide, N-propylacrylamide, N-isopropylacrylamide, N-butylacrylamide, N,N-dimethylacrylamide, N,N-diethylacrylamide, N,N-dipropylacrylamide, N,N-dibutylacrylamide, N,N-methylethylacrylamide, N,N-methylpropylacrylamide, N,N-ethylpropylacrylamide, N,N-ethylbutylacrylamide, N,N-propylbutylacrylamide, N-cyclopropylacrylamide, N-cyclobutylacrylamide, N-vinylpyrrolidone and the like and mixtures thereof. In one embodiment, the acrylamido-containing monomers are hydrophilic monomers.

The polymeric material for use in forming the core containing the pharmaceutically active agents of the drug delivery devices of the present invention can be a crosslinked polymeric network. Preferably, the crosslinking agent is one that is copolymerized with the reactive monomers. Suitable crosslinking agents include, but are not limited to, any di- or multi-functional crosslinking agent and the like and mixtures thereof. Representative examples of such crosslinkers include, but are not limited to, tripropylene glycerol diacrylate, ethylene glycol dimethacrylate, tetraethylene glycol dimethacrylate, poly(ethylene glycol diacrylate) (PEG400 or PEG600), methylene bis acrylamide and the like and mixtures thereof. If used, the crosslinking agent is used in an effective amount, by which is meant an amount that is sufficient to cause crosslinking of the monomeric mixture resulting in a copolymer capable of being combined with the one or more pharmaceutically active agents such as entrapping the one or more pharmaceutically active agents to produce the desired core of the drug delivery device. The amount of the crosslinking agent can range from about 0.05% w/w to about 20% w/w and preferably from about 0.1% w/w to about 10% w/w.

In general, the copolymerization reaction can be conducted neat, that is, the monomeric mixture and optional crosslinking agent(s) are combined in the desired ratio, and then exposed to, for example, ultraviolet (UV) light or electron beams in the presence of one or more photoinitiator(s) or at a suitable temperature, for a time period sufficient to form the copolymer. As discussed hereinbelow, copolymerization can be carried out in the presence of the one or more pharmaceutically active agents. Alternatively, the one or more pharmaceutically active agents can be combined with the first polymeric material after polymerization has been carried out by techniques known in the art, e.g., solvent entrapment method, thermal polymerization and the like. Suitable reaction times will ordinarily range from about 1 minute to about 24 hours and preferably from about 1 hour to about 4 hours.

The use of UV or visible light in combination with photoinitiators is well known in the art and is particularly suitable for formation of the copolymer. Numerous photoinitiators of the type in question here are commercial products. Photoinitiators enhance the rapidity of the curing process when the photocurable compositions as a whole are exposed to, for example, ultraviolet radiation. Suitable photoinitiators which are useful for polymerizing the polymerizable mixture of monomers can be commercially available photoinitiators. They are generally compounds which are capable of initiating the radical reaction of olefinically unsaturated double bonds on exposure to light with a wavelength of, for example, about 260 to about 480 mn.

Examples of suitable photoinitiators for use herein include, but are not limited to, one or more photoinitiators commercially available under the “IRGACURE”, “DAROCUR” and “SPEEDCURE” trade names (manufactures by Ciba Specialty Chemicals, also obtainable under a different name from BASF, Fratelli Lamberti and Kawaguchi), e.g., “IRGACURE” 184 (1-hydroxycyclohexyl phenyl ketone), 907 (2-methyl-1-[4-(methylthio)phenyl]-2-morpholino propan-1-one), 369 (2-benzyl-2-N,N-dimethylamino-1-(4-morpholinophenyl)-1-butanone), 500 (the combination of 1-hydroxy cyclohexyl phenyl ketone and benzophenone), 651 (2,2-dimethoxy-2-phenyl acetophenone), 1700 (the combination of bis(2,6-dimethoxybenzoyl-2,4,4-trimethyl pentyl)phosphine oxide and 2-hydroxy-2-methyl-1-phenyl-propan-1-one), and 819 [bis(2,4,6-trimethyl benzoyl)phenyl phosphine oxide] and “DAROCUR” 1173 (2-hydroxy-2-methyl-1-phenyl-1-propan-1-one) and 4265 (the combination of 2,4,6-trimethylbenzoyldiphenyl-phosphine oxide and 2-hydroxy-2-methyl-1-phenyl-propan- 1-one); and the like and mixtures thereof. Other suitable photoimtiators for use herein include, but are not limited to, alkyl pyruvates such as methyl, ethyl, propyl, and butyl pyruvates, and aryl pyruvates such as phenyl, benzyl, and appropriately substituted derivatives thereof. Generally, the amount of photoinitiator can range from about 0.05% w/w to about 5% w/w and preferably from about 0.1% w/w to about 1% w/w.

Copolymerization of the foregoing monomeric mixtures and optional crosslinking agent(s) can be carried out in any known manner. The important factors are intimate contact of the reactive monomers in, for example, the presence of the photoinitiator(s). The components in the reaction mixture can also be added continuously to a stirred reactor or can take place in a tubular reactor in which the components can be added at one or more points along the tube. Generally, in one embodiment the acrylate ester and/or methacrylate ester-containing monomer(s) can be added to a reaction mixture in an amount ranging from about 10% w/w to about 80% w/w and preferably from about 20% w/w to about 50% w/w and the acrylamido-containing monomer(s) can be added to the reaction mixture in an amount ranging from about 90% w/w to about 10% w/w and preferably from about 80% w/w to about 30% w/w.

In an alternative embodiment, the process may include at least polymerizing the monomeric mixture in the presence of one or more pharmaceutically active agents under polymerization conditions as discussed above such that the pharmaceutically active agent(s) is entrapped in the polymerization product. In this embodiment, it is particularly advantageous to carry out the polymerization process by exposing the monomeric mixture and pharmaceutically active agent(s) to UV or visible light in the presence of one or more photoinitiator(s). As one skilled in the art will readily appreciate, the resulting polymerization product may have some pharmaceutically active agent(s) which is covalently bound to the polymerization product as well as some free starting monomer(s). If desired, these reactants can be removed as discussed hereinbelow.

The shell of the drug delivery device of the present invention is then formed over the core and encapsulates the core. A wide variety of materials which are permeable to passage of the one or more pharmaceutically active agents may be used as a second polymeric material for forming the shell of the drug delivery systems of the present invention. The only requirements are that they are inert, non-immunogenic, of the desired permeability, and capable of being cut into shaped articles. Materials that may be suitable for fabricating the shell include naturally occurring or synthetic materials that are biologically compatible with body fluids and body tissues, and essentially insoluble in the body fluids with which the material will come in contact and capable of being cut into shaped articles. Exemplary polymeric materials include those prepared by reacting a monomeric mixture containing at least one or more acrylate ester and/or methacrylate ester-containing monomers and one or more acrylamido-containing monomers optionally in the presence of one or more crosslinking agents and can be any of the acrylate ester and/or methacrylate ester-containing monomers, acrylamido-containing monomers and crosslinking agents described hereinabove. Representative examples of other monomers for use in a monomeric mixture to be polymerized include, but are not limited to, silicones, urethanes, carbamates, polyesters, polyimines and the like and mixtures thereof. The resulting copolymers can be in random or block sequences.

In one embodiment, the first and second polymeric material are the same material. In another embodiment, the first and second polymeric material are different material. The shell can be prepared according to polymerization conditions described hereinabove with respect to the core.

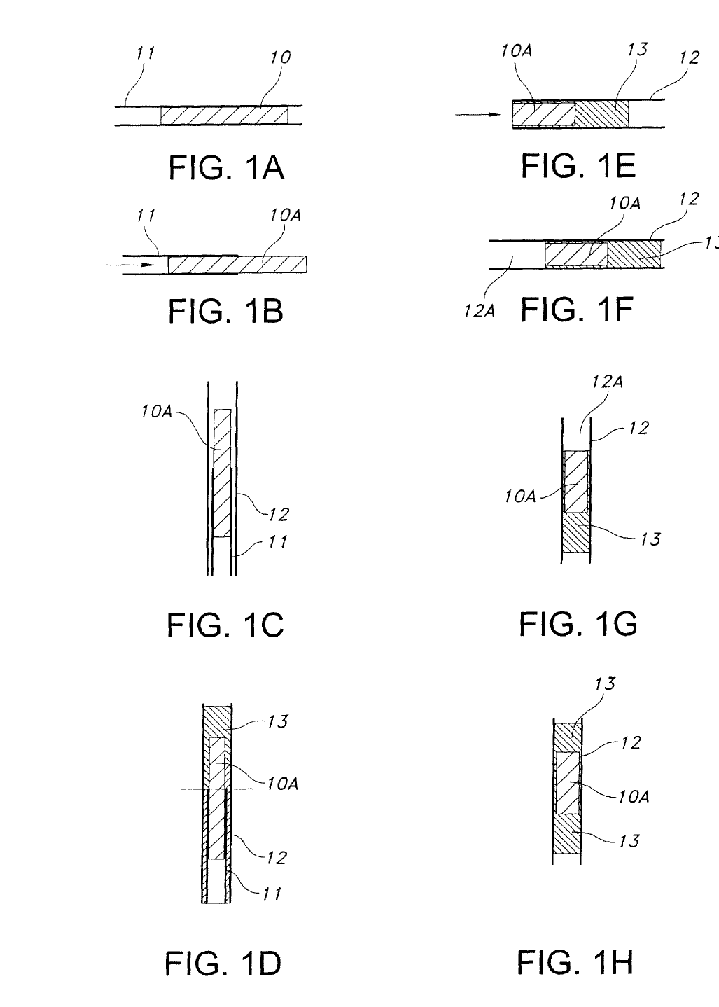

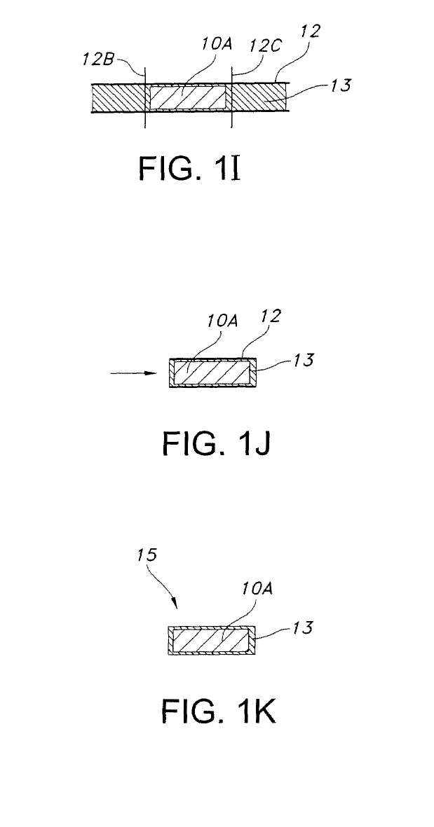

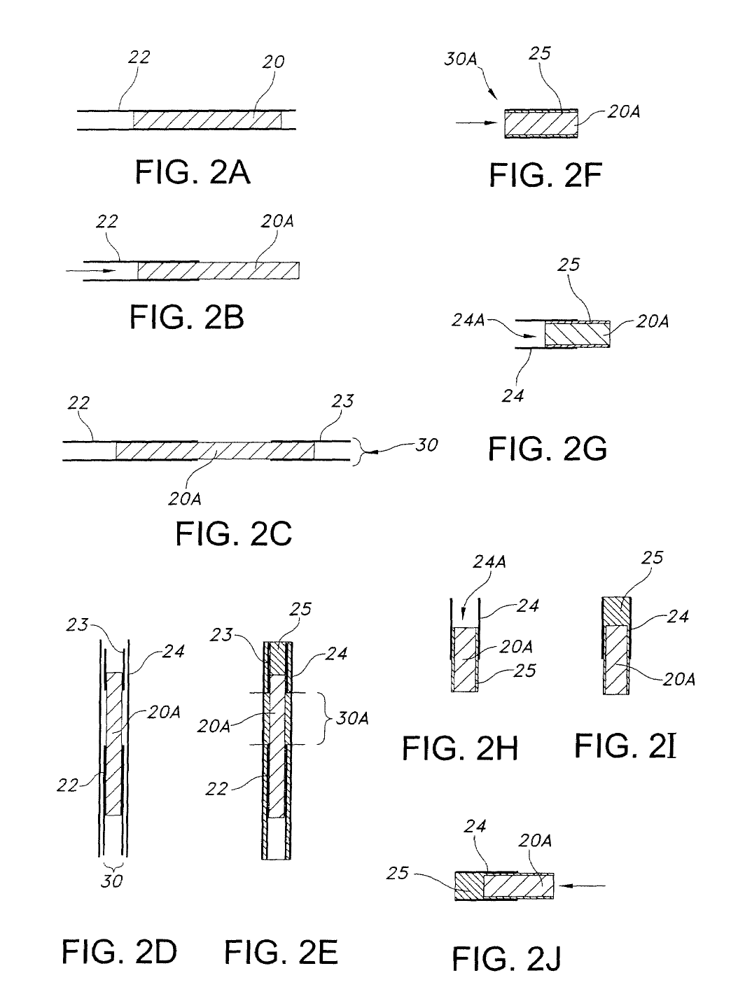

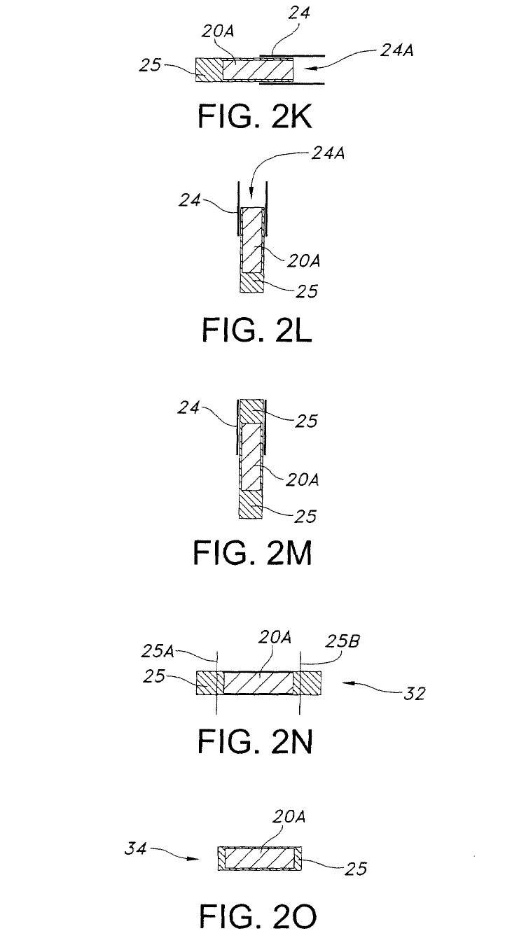

The drug delivery devices of the present invention can be prepared by techniques known in the art. In one embodiment, the drug delivery devices of the present invention can be made as generally shown in FIGS. 1 and 2 and as exemplified in the examples herein. The drug delivery devices of the present invention may be manufactured in any suitable form, shape, e.g., circular, rectangular, tubular, square and triangular shapes, or size suitable for the treatment which they are intended to be used. It will be appreciated the dimensions of the device including at least the core and shell surrounding the core can vary with the size of the device, the size of the core, and the size of the shell. The physical size of the device should be selected so that it does not interfere with physiological functions at the implantation site of the mammalian organism. The targeted disease state, type of mammalian organism, location of administration, and agents or agent administered are among the factors which would effect the desired size of the drug delivery device. However, because the device is intended for placement in the eye, the device is relatively small in size. In one embodiment, the drug delivery device is sized and configured for implantation or injection in eye tissue. Generally, the device can have a maximum height, width and length each no greater than about 10 mm, preferably no greater than about 5 mm, and most preferably no greater than about 3 mm.

Following the formation of the drug delivery device, the drug delivery device is extracted with supercritical fluid to remove residual materials therefrom. For example, in the case of a polymeric core and shell, the core and/or shell may include one or more contaminants such as lower molecular weight materials, e.g., unreacted monomeric material and oligomers. Preferably, the drug delivery device comprises a pharmaceutically active salt, and the contaminants are hydrophobic, such as unreacted hydrophobic monomers, e.g., alkyl methacrylates, alkyl methacrylamides, silicone based prepolymers, etc. Such materials may irritate eye tissue. Generally, traditional extracting solvents do not lend themselves to extracting devices already containing pharmaceutically active agent, as relatively large amounts of various pharmaceutically active agents would be dissolved in and removed by the traditional extracting solvents such as isopropanol and similar solvents. Accordingly, the drug delivery device obtained herein will be contact with at least supercritical fluid (SCF) to extract the one or more contaminants after the device is loaded with the active agent.

As mentioned, any pharmaceutically acceptable form of the pharmaceutically active agent may be employed in this invention. However, many SCFs, including supercritical carbon dioxide, are relatively hydrophobic. Thus, the SCF can better dissolve hydrophobic material. Accordingly, this invention can be particularly useful in extracting hydrophobic contaminants. The salt forms of various pharmaceutically active agents are relatively hydrophilic and therefore this invention can be useful in extracting devices containing pharmaceutically active salts, in that the active salts are not readily dissolved in, nor removed from the device by, the treatment with at least supercritical fluid.

If desired, it is possible to modify the solubility of a pharmaceutical active agent (hydrophobic or hydrophilic) in the supercritical fluid by changing such conditions as pressure and/or temperature and by using an appropriate co-solvent in the supercritical fluid, e.g., CO₂. For example, by using a small concentration (e.g., about 1 to about 10 wt. %) of a polar or protic co-solvent, the supercritical fluid can be made more polar and hydrophobic oligomeric or unreacted monomeric contaminants with low vapor pressures can be preferentially dissolved from the drug delivery device into the supercritical fluid without removing the hydrophobic pharmaceutical active agent. Alternately, when polar or water soluble pharmaceutical active agents are present in the drug delivery device, using only non-polar solvents as supercritical fluids, the hydrophobic contaminants can be selectively extracted. Suitable polar or protic co-solvents include, but are not limited to, ketones, e.g., acetone and the like, alcohols, e.g., ethanol and the like, and mixtures thereof.

The drug delivery devices of the present invention may be used in a broad range of therapeutic applications. The drug delivery systems of the present invention are particularly useful in the treatment of an ophthalmic state, disease, disorder, injury or condition. Representative examples of such an ophthalmic state, disease, disorder, injury or condition include, but are not limited to, diabetic retinopathy, glaucoma, macular degeneration, retinitis pigmentosa, retinal tears or holes, retinal-detachment, retinal ischemia, acute retinopathies associated with trauma, inflammatory mediated degeneration, post-surgical complications, damage associated with laser therapy including photodynamic therapy (PDT), surgical light induced iatrogenic retinopathy, drug-induced retinopathies, autosomal dominant optic atrophy, toxic/nutritional amblyopias; leber's hereditary optic neuropathy (LHOP), other mitochondrial diseases with ophthalmic manifestations or complications, angiogenesis; atypical RP; bardet-biedl syndrome; blue-cone monochromacy; cataracts; central areolar choroidal dystrophy; choroideremia; cone dystrophy; rod dystrophy; cone-rod dystrophy; rod-cone dystrophy; congenital stationary night blindness; cytomegalovirus retinitis; diabetic macular edema; dominant drusen; giant cell arteritis (GCA); goldmann-favre dystrophy; graves' ophthalmopathy; gyrate atrophy; hydroxychloroquine; iritis; juvenile retinoschisis; kearns-sayre syndrome; lawrence-moon bardet-biedl syndrome; leber congenital amaurosis; lupus-induced cotton wool spots; macular degeneration, dry form; macular degeneration, wet form; macular drusen; macular dystrophy; malattia leventinese; ocular histoplasmosis syndrome; oguchi disease; oxidative damage; proliferative vitreoretinopathy; refsum disease; retinitis punctata albescens; retinopathy of prematurity; rod monochromatism; RP and usher syndrome; scleritis; sector RP; sjogren-larsson syndrome; sorsby fundus dystrophy; stargardt disease and other retinal diseases.

The drug delivery devices can be administered to a mammal in need of treatment by way of a variety of routes. For example, the drug delivery devices may be used by implantation within a portion of the body in need of localized drug delivery, e.g., the drug delivery device may be implanted below the sclera. Alternately, the device may be implanted by injecting the device into the eye. For example, a sphere- or cylinder-shaped device may be inserted into the vitreous through a 0.5-mm opening in the sclera provided by a TSV-25 cannula. However, the subject drug delivery devices may likewise be used in accordance with other surgical procedures known to those skilled in the field of ophthalmology. For example, the drug delivery systems can be administered to the region of the eye in need of treatment employing instruments known in the art, e.g., a flexible microcatheter system or cannula disclosed in U.S. Patent Application Publication No. 2002/0002362, or the intraretinal delivery and withdrawal systems disclosed in U.S. Pat. Nos. 5,273,530 and 5,409,457, the contents of each which are incorporated by reference herein. The pharmaceutically active agent may be released from the drug delivery device over a sustained and extended period of time.

The following examples are provided to enable one skilled in the art to practice the invention and are merely illustrative of the invention. The examples should not be read as limiting the scope of the invention as defined in the claims.

### EXAMPLE 1

A drug delivery device according to the present invention can be made as follow:

Step 1. Inject a suitable solution 10 of a monomer mixture with a high load of drug dissolved in it into a 0.25 mm Inner Diameter (ID), 0.45 mm Outer Diameter (OD) fluoropolymer tubing 11 and clamp off both ends of tubing 11 (not shown). The drug-loaded monomer mixture 10 is polymerized with a 2-hour cure under UV light (See FIG. 1A).

Step 2. An approximately 3 cm length of tubing 11 containing cured drug-loaded polymer core 10 is cut. An approximately 1 cm length of the drug-loaded polymer core 10A of drug-loaded polymer core 10 is exposed at one end by pushing the drug-loaded polymer core 10 partway out of tubing 11 (See FIG. 1B).

Step 3. An approximately 5 cm piece of larger diameter (0.5 mm ID, 0.75 mm OD) fluoropolymer tubing 12 is held in a vertical position with a clamp at its base (not shown). The small diameter tubing 11 with partially exposed drug-loaded polymer core 10A is inserted into larger diameter tubing 12, such that exposed drug-loaded polymer core 10A is facing up (See FIG. 1C).

Step 4. A monomer mixture 13 containing no drug is injected into larger diameter tubing 12 such that it surrounds the exposed drug-loaded polymer core 10A and tubing 11. The small diameter tubing 11 acts as a spacer, keeping the drug-loaded polymer 10A centered in larger diameter tubing 12. The drug free monomer mixture 13 is cured under UV light for 2 hours (See FIG. 1D).

Step 5. The larger diameter tubing 13 is cut at the end of inner tubing 11 (See FIGS. 1D-1E).

Step 6. The large diameter polymer tubing 12 containing the drug-loaded polymer core 10A and drug-free polymer shell 13 is pushed partway through the large diameter tubing to expose 1 cm of open tubing 12A at the end of tubing 12 (See FIGS. 1E-1F).

Step 7. The large diameter tubing is clamped in a vertical position with the open end 12A of tubing 12 at the top (FIG. 1G). A drug free monomer mixture 13 is injected into the open end 12A of tubing 12 and on top of the drug-loaded polymer core 10A. Drug free monomer mixture 13 is cured under UV light for 2 hours (See FIG. 1H).

Step 8. The large diameter polymer tubing 12 containing drug-free polymer shell 13 is cut 0.125 mm outside the ends 12B and 12C and the tubing 12 containing the drug-loaded polymer core 10A and drug-free polymer shell 13 is removed from tubing 12 (FIGS. 1I-1J). This leaves the finished drug delivery device 15 consisting of drug-loaded polymer core 10A (0.25 mm diameter) surrounded by drug-free polymer shell 13 (0.125 mm thickness) (FIG. 1K).

The device is extracted with supercritical fluid, such as supercritical carbon dioxide. The exposure to supercritical fluid removes contaminants, including unreacted monomers or oligomers present in the device.

### EXAMPLE 2

A drug delivery device according to the present invention can be made as follow:

Step 1. Inject a suitable solution 20 of a monomer mixture with a high load of drug dissolved in it into 0.25 mm ID, 0.45 mm OD fluoropolymer tubing 22, and clamp off both ends of tubing 22 (not shown). The drug-loaded monomer mixture 20 is polymerized with a 2 hour cure under UV light (See FIG. 2A).

Step 2. An approximately 5 cm length of tubing 22 containing cured drug-loaded polymer core 20 is cut. An approximately 3 cm length of the drug-loaded polymer 20A is exposed at one end by pushing the drug-loaded polymer core 20 partway out of tubing 22 (See FIG. 2B) to expose drug-loaded polymer core 20A.

Step 3. An approximately 2 cm piece of empty 0.25 mm ID fluoropolymer tubing 23 is pushed onto the exposed drug-loaded polymer core 20A, leaving 1 cm of exposed drug-loaded polymer core 20A between two 2 cm long portions of drug loaded polymer core covered by tubing 22 and 23 (See FIG. 2C) to provide tubing 30.

Step 4. A 5 cm piece of larger diameter (0.5 mm ID, 0.75 mm OD) fluoropolymer tubing 24 is held in a vertical position with a clamp at its base. Tubing 30 containing the drug-loaded polymer core is inserted into the larger diameter tubing 24 (See FIG. 2D).

Step 5. A monomer mixture 25 containing no drug is injected into the large diameter tubing 24 such that it surrounds the tubing 30. The small diameter tubing 22 and 23 at both ends of tubing 24 act as a spacer, keeping the exposed drug-loaded polymer core 30A centered in large diameter tubing 24. The drug free monomer mixture is cured under UV light for 2 hours (See FIG. 2E).

Step 6. The large diameter polymer tubing 24 is cut at both ends of tubing 22 and 23 (See FIGS. 2E-2F) to provide tubing 30A.

Step 7. The drug-loaded polymer 30A is pushed partway through the large diameter tubing 24 to expose approximately 0.5 cm of open tubing 24A at one of the ends (See FIGS. 2F-2G).

Step 8. The large diameter tubing 24 is clamped in a vertical position with the open end 24A of tubing 24 at the top. Drug-free monomer mixture 25 is injected into the open end 24A of tubing 24 and cured under UV light for 2 hours (See FIGS. 2H-21).

Step 9. Steps 7 and 8 are repeated in the opposite direction to make a drug-free polymer shell 25 at the other end of the drug-loaded polymer core 20A (See FIGS. 2J-2M).

Step 10. The resulting rod 32 is cut 0.125 mm outside the ends 25A and B of the drug-loaded polymer core 20A and removed from tubing 24 (FIG. 2N). This leaves the finished drug delivery device 34 containing a drug-loaded polymer core 20A (0.25 mm diameter) surrounded by a drug-free polymer shell 25 (0.125 mm thickness) (See FIG. 20).

The device is extracted with supercritical fluid, such as supercritical carbon dioxide. The exposure to supercritical fluid removes contaminants, including unreacted monomers or oligomers present in the device.

It will be understood that various modifications may be made to the embodiments disclosed herein. Therefore the above description should not be construed as limiting, but merely as exemplifications of preferred embodiments. For example, while there is shown and described herein monomers, copolymers, matrix controlled diffusion drug delivery systems and methods of making and using the same, it will be manifest to those skilled in the art that various modifications may be made without departing from the spirit and scope of the underlying inventive concept. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A method for making an ocular drug delivery device, the method comprising providing a drug delivery device comprising (a) a core comprising a therapeutically effective amount of one or more pharmaceutically active agents and a first polymeric material, and a shell covering the core, the shell comprising a second polymeric material which is permeable to passage of the one or more pharmaceutically active agents, wherein the first and/or second polymeric material include one or more contaminants and wherein the drug delivery device is sized and configured for implantation or injection in eye tissue; and (b) subjecting the drug delivery device to a supercritical fluid to remove the contaminants.

2. The method of claim 1, wherein the first polymeric material and the second polymeric material are the same material.

3. The method of claim 1, wherein the first polymeric material and the second polymeric material are different material.

4. The method of claim 1, wherein the first polymeric material comprises a reaction product of a monomeric mixture comprising one or more acrylate ester and/or methacrylate ester-containing monomers and one or more acrylamido-containing monomers.

5. The method of claim 4, wherein the one or more acrylate ester and/or methacrylate ester-containing monomers is represented by general formula I: 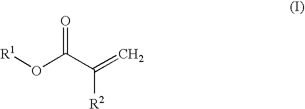
wherein R¹ is a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, C₁-C₁₈ alkyl siloxysilane, C₁-C₁₈ alkyl siloxane, an ether or polyether containing group, substituted or unsubstituted, linear or branched, and R² is H or CH₃.

6. The method of claim 4, wherein the one or more acrylate ester and/or methacrylate ester-containing monomers is selected from the group consisting of a methyl acrylate, ethyl acrylate, propyl acrylate, isopropyl acrylate, n-butyl acrylate, iso-butyl acrylate, t-butyl acrylate, n-hexyl acrylate, 2-ethylbutyl acrylate, 2-ethylhexyl acrylate, cyclopropyl acrylate, cyclobutyl acrylate, cyclohexyl acrylate, benzyl acrylate, 2-phenoxyethyl acrylate, phenyl acrylate, 2-phenylethyl acrylate, 3-phenylpropyl acrylate, 3-phenoxypropyl acrylate, 4-phenylbutyl acrylate, 4-phenoxybutyl acrylate, 4-methylphenyl acrylate, 4-methylbenzyl acrylate, 2-2-methylphenylethyl acrylate, 2-3-methylphenylethyl acrylate, 2-methylphenylethyl acrylate and mixtures thereof.

7. The method of claim 4, wherein the one or more acrylamido-containing monomers is represented by the general formulae II and III: 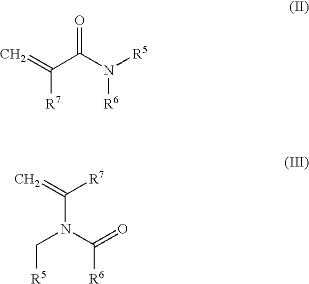
wherein R⁵ and R⁶ are independently hydrogen, a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, C₁-C₁₈ alkyl siloxysilane, or C₁-C₁₈ alkyl siloxane, substituted or unsubstituted, linear or branched, or R⁵ and R⁶ together with the nitrogen atom to which they are bonded are joined together to form a heterocyclic group and R⁷ is H or CH₃.

8. The method of claim 4, wherein the one or more acrylamido-containing monomer is selected from the group consisting of acrylamide, N-methylacrylamide, N-ethylacrylamide, N-propylacrylamide, N-isopropylacrylamide, N-butylacrylamide, N,N-dimethylacrylamide, N,N-diethylacrylamide, N,N-dipropylacrylamide, N,N-dibutylacrylamide, N,N-methylethylacrylamide, N,N-methylpropylacrylamide, N,N-ethylpropylacrylamide, N,N-ethylbutylacrylamide, N,N-propylbutylacrylamide, N-cyclopropylacrylamide, N-cyclobutylacrylamide and mixtures thereof.

9. The method of claim 4, wherein the monomeric mixture further comprises one or more crosslinking agents.

10. The method of claim 9, wherein the crosslinking agent is selected from the group consisting of tripropylene glycerol diacrylate, ethylene glycol dimethacrylate, tetraethylene glycol dimethacrylate, poly(ethylene glycol diacrylate), methylene bis acrylamide and mixtures thereof.

11. The method of claim 1, wherein the second polymeric material comprises a reaction product of a monomeric mixture comprising one or more acrylate ester and/or methacrylate ester-containing monomers and one or more acrylamido-containing monomers.

12. The method of claim 11, wherein the one or more acrylate ester and/or methacrylate ester-containing monomers is represented by general formula I: 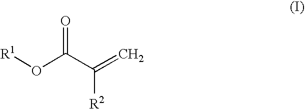
wherein R¹ is a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, C₁-C₁₈ alkyl siloxysilane, C₁-C₁₈ alkyl siloxane, an ether or polyether containing group, substituted or unsubstituted, linear or branched, and R² is H or CH₃.

13. The method of claim 1 1, wherein the one or more acrylate ester and/or methacrylate ester-containing monomers is selected from the group consisting of a methyl acrylate, ethyl acrylate, propyl acrylate, isopropyl acrylate, n-butyl acrylate, iso-butyl acrylate, t-butyl acrylate, n-hexyl acrylate, 2-ethylbutyl acrylate, 2-ethylhexyl acrylate, cyclopropyl acrylate, cyclobutyl acrylate, cyclohexyl acrylate, benzyl acrylate, 2-phenoxyethyl acrylate, phenyl acrylate, 2-phenylethyl acrylate, 3-phenylpropyl acrylate, 3-phenoxypropyl acrylate, 4-phenylbutyl acrylate, 4-phenoxybutyl acrylate, 4-methylphenyl acrylate, 4-methylbenzyl acrylate, 2-2-methylphenylethyl acrylate, 2-3-methylphenylethyl acrylate, 2-methylphenylethyl acrylate and mixtures thereof.

14. The method of claim 1 1, wherein the one or more acrylamido-containing monomers is represented by the general formulae II and III: 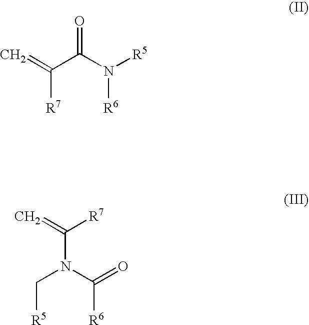
wherein R⁵ and R⁶ are independently hydrogen, a C₁-C₁₈ alkyl, C₃-C₁₈ cycloalkyl, C₃-C₁₈ cycloalkylalkyl, C₃-C₁₈ cycloalkenyl, C₅-C₃₀ aryl, C₅-C₃₀ arylalkyl, C₁-C₁₈ alkyl siloxysilane, or C₁-C₁₈ alkyl siloxane, substituted or unsubstituted, linear or branched, or R⁵ and R⁶ together with the nitrogen atom to which they are bonded are joined together to form a heterocyclic group and R⁷ is H or CH₃.

15. The method of claim 11, wherein the one or more acrylamido-containing monomer is selected from the group consisting of acrylamide, N-methylacrylamide, N-ethylacrylamide, N-propylacrylamide, N-isopropylacrylamide, N-butylacrylamide, N,N-dimethylacrylamide, N,N-diethylacrylamide, N,N-dipropylacrylamide, N,N-dibutylacrylamide, N,N-methylethylacrylamide, N,N-methylpropylacrylamide, N,N-ethylpropylacrylamide, N,N-ethylbutylacrylamide, N,N-propylbutylacrylamide, N-cyclopropylacrylamide, N-cyclobutylacrylamide and mixtures thereof.

16. The method of claim 1, wherein the one or more pharmaceutically active agents is selected from the group consisting of an anti-glaucoma agent, anti-cataract agent, anti-diabetic retinopathy agent, thiol cross-linking agent, anti-cancer agent, immune modulator agent, anti-clotting agent, anti-tissue damage agent, anti-inflammatory agent, anti-fibrous agent, non-steroidal anti-inflammatory agent, antibiotic, anti-pathogen agent, piperazine derivative, cycloplegic agent, miotic agent, mydriatic agent and mixtures thereof.

17. The method of claim 1, wherein the one or more pharmaceutically active agents is selected from the group consisting of an anticholinergic, anticoagulant, antifibrinolytic, antihistamine, antimalarial, antitoxin, chelating agent, hormone, immunosuppressive, thrombolytic, vitamin, protein, salt, desensitizer, prostaglandin, amino acid, metabolite, antiallergenic and mixtures thereof.

18. The method of claim 1, wherein the drug delivery device comprises a pharmaceutically active salt, and the contaminants are hydrophobic.

19. The method of claim 1, wherein the supercritical fluid is selected from the group consisting of supercritical carbon dioxide, supercritical nitrous oxide, supercritical ethane and supercritical propane.

20. The method of claim 1, wherein the supercritical fluid comprises supercritical carbon dioxide.

21. A method for making an ocular drug delivery device, the method comprising providing a drug delivery device comprising (a) a core comprising a therapeutically effective amount of one or more pharmaceutically active agents and a first polymeric material, and (b) a shell covering the core, the shell comprising a second polymeric material which is permeable to passage of the one or more pharmaceutically active agents; and removing contaminants from the device by subjecting the device to a supercritical fluid.
